SEQUENCE LISTING (8 sequences)

SEQ ID NO: 1 - DNA, 28 bp, Artificial Sequence
  Primer GAP#1
gcggccgctggtgggtgcatgtatgtac

SEQ ID NO: 2 - DNA, 26 bp, Artificial Sequence
  Primer GAP#5
cccggggatggtaagagtgttagaga

SEQ ID NO: 3 - DNA, 33 bp, Artificial Sequence
  Primer GAP#33
ggatccgtcgactaaacggttctctccaaaccc

SEQ ID NO: 4 - DNA, 28 bp, Artificial Sequence
  Primer GAP#4
ggtaccttgatcagataaagatagagat

SEQ ID NO: 5 - DNA, 20 bp, Artificial Sequence
  Primer R#1
gagctctcgagtggacggtg

SEQ ID NO: 6 - DNA, 28 bp, Artificial Sequence
  Primer R#2
gcggccgcgagctcatcccgcttcactc

SEQ ID NO: 7 - DNA, 12 bp, Artificial Sequence
  BamHI-SalI site
ggatccgtcgac

SEQ ID NO: 8 - DNA, 14 bp, Artificial Sequence
  NotI-SacI site
gcggccgcgagctc

(19) United States
(12) Patent Application Publication
(10) Pub. No.: US 2006/0141557 A1
(43) Pub. Date: Jun. 29, 2006

(54) Production of canthaxanthin by phaffia

(76) Inventors: Tatsuo Hoshino, Kamakura-shi (JP); Kazuyuki Ojima, Fujisawa-shi (JP); Yutaka Setoguchi, Fujisawa-shi (JP)

Correspondence Address: Stephen M Haracz;Bryan Cave, 1290 Avenue of the Americas, New York, NY, 10104, US

(21) Appl. No.: 10/528,846

(22) PCT Filed: Sep. 16, 2003
(86) PCT No.: PCT/EP03/10294

§ 371 Date: Feb. 9, 2006

(30) Foreign Application Priority Data
Sep. 27, 2002 (EP) 02021600.8

(51) Int. Cl. C12P 23/00; C12N 1/20

(52) U.S. Cl. 435/67; 435/252.2

(57) ABSTRACT

Disclosed is a process for producing canthaxanthin and echinenone which comprises cultivating a recombinant microorganism which is expressing a β-casotene ketolase gene and belonging to the genus Xanthophyllomyces (Phaffia) in an aqueous nutrient medium under aerobic conditions, and isolating the resulted carotenoids from the cells of said recombinant microorganism or from the cultured broth.

The present invention relates to production of xanthophyll carotenoids, especially canthaxanthin and echinenone by a microorganism belonging to the genus Phaffia.

More particularly, the present invention provides a process for producing xanthophyll carotenoids, especially canthaxanthin and echinenone by genetically modified recombinant microorganism belonging to the genus Phaffia.

By using methods of the present invention, it becomes possible to produce the useful xanthophyll carotenoids other than astaxanthin such as canthaxanthin and echinenone as a major xanthophyll carotenoid in the recombinant microorganism belonging to Phaffia.

Any strain which is able to produce β-carotene can be a suitable host strain. When the selected host strain is a normal Phaffia strain which is capable of producing astaxanthin from β-carotene, production of mixture of astaxanthin and other xanthophyll carotenoids can be achieved after introduction and expression of a gene coding for β-carotene ketolase. On the other hand, when a strain which cannot produce astaxanthin and is accumulating β-carotene is selected as the said host strain, it is expected to produce maximum level of xanthophyll carotenoids such as canthaxanthin and echinenone without accumulation of astaxanthin. Such a host strain which accumulates β-carotene can be obtained by mutagenesis of a strain of Phaffia rhodozyma accumulating astaxanthin. Alternatively, such a host strain accumulating β-carotene can also be obtained by deactivating astaxanthin synthase which is an enzyme involved in the biosynthesis of astaxanthin from β-carotene and is disclosed in U.S. Pat. No. 6,365,386. Disruption of the gene of astaxanthin synthase will be one of the most convenient ways to deactivate the enzyme.

Furthermore, such a host strain accumulating β-carotene can also be obtained from public type culture collections. For example, P. rhodozyma ATCC96815 accumulating β-carotene can be purchased Srom American Type Culture Collection (P.O.Box 1549, Manassas, Va. 20108, USA). Isolating a new β-carotene accumulating strain of P. rhodozyma, which may be a derivative or a spontaneous mutant of astaxanthin producing strain, from nature will be another approach to prepare the host strain of the present invention.

An aspect of the present invention is a process for producing canthaxanthin and echinenone which comprises cultivating a recombinant Phaffia strain which is expressing the β-carotene ketolase.

Said β-carotene ketolase catalyzes conversion of methylene to keto groups at positions 4 and 4′ on the β-ionone ring of β-carotene to produce the xanthophyll, canthaxanthin via echinenone. Genes encoding this enzyme have been isolated from several species, for example, crtW from marine bacteria (Agrobacterium aurantiacuin and Alcaligenes sp.), crtw from Paracoccus marcusii (GenBank accession No. Y15112), crtW from Paracoccus carotinifaciens sp.nov., and bkt gene of Haematococcus pluvialis (GenBank accession No. D45881).

In the present invention, any gene encoding a protein having the β-carotene ketolase activity can be used.

Preferably, said β-carotene ketolase gene can be obtained from a microorganism which is selected from the group consisting of microorganisms of the genera Agrobacterium, Alcaligenes, Paracoccus, and Haematococcus having the β-carotene ketolase gene.

More preferably, said β-carotene ketolase gene can be obtained from a microorganism which is selected from the group consisting of Agrobacterium aurantiacum (GenBank accession No. D58420), Alcaligenes PC-1 (GenBank accession No. D58422), Paracoccus marcusii MH1 (GenBank accession No. Y15112), a gram-negative bacteria E-396 (FERM BP-4283) (the DNA sequence of the β-carotene ketolase gene originated from this microorganism can be seen in the description of JP-A Hei 10-155497), and Haematococcus pluvialis (GenBank accession No. D45881) which are having the β-carotene ketolase gene, the GenBank accession No. showing the DNA sequence of the β-carotene ketolase gene originated from the respective microorganism.

Still more preferably, said β-carotene ketolase gene can be obtained from Alcaligenes PC-1, or it can also be obtained as a DNA sequence which is substantially homologous thereto.

The expression “a DNA sequence which is substantially homologous” refers with respect to the DNA sequence encoding the β-carotene ketolase to a DNA sequence which encodes an amino acid sequence which shows more than 60%, preferably more than 70%, more preferably more than 80%, and most preferably more than 90% identical amino acids when compared to the amino acid sequence of crtW of Alcaligenes PC-1 and is the amino acid sequence of a polypeptide which shows the same type of enzymatic activity as the enzyme encoded by crtW of Alcaligenes PC-1.

By using the β-carotene ketolase gene, it is possible to render a microorganism belonging to genus Phaffia an ability to produce canthaxanthin and echinenone. The recombinant microorganism of Phaffia expressing a β-carotene ketolase gene can be prepared by the well-known recombinant technology.

The techniques used to isolate or clone a DNA encoding β-carotene ketolase of the present invention are known in the art and include isolation from genomic DNA. The cloning of the DNA sequence of the present invention from such genomic DNA can be effected by using the polymerase chain reaction (hereinafter referred to as PCR).

The isolated or cloned DNA encoding β-carotene ketolase can be preferably utilized after cloning on a suitable expression vector for expression of the enzyme in the host microorganism of Phaffia.

“Expression vector” includes vectors which are capable of expressing DNA sequences contained therein where such sequences are operably linked to other sequences such as control sequences which are capable of effecting the expression of said DNA sequences in a microorganism belonging to genus Phaffia. The term “operably linked” refers to a juxtaposition wherein the components so described are in a relationship permitting them to function in their intended manner. The term “control sequence” is intended to include, at a minimum, components which are necessary for expression of the gene of interest, and may also include additional advantageous components. Generally the control sequences include promoters, terminators and, in some instances, enhancers, transactivators, or transcription factors. Constitutive promoters, such as the glyceraldehyde-3-dehydrogenase (GAP) gene promoter derived from P. rhodozyma (WO 97/23,633), may be used to obtain constitutive expression. Inducible promoters can be also used in order to achieve an exactly-controlled expression. One of the examples for the inducible promoters is the promoter of genes encoding heat shock proteins or amylase gene, and the like.

Methods which are well known to those skilled in the art may be used to construct said expression vectors.

It is implied, although not explicitly stated, that expression vectors must be replicable in the host organisms either as episomes or as an integral part of a chromosomal DNA. Generally, higher stability of the gene may be expected in the latter case. To integrate the expression vector into the chromosome of the host microorganism by a homologous recombination, a vector is prepared to contain at least a portion of the DNA fragment whose sequence is homologous to the host genomic DNA. For this purpose, rDNA gene fragment can be effectively used in a microorganism belonging to Phaffia. The rDNA is a kind of satellite DNAs which exist in multicopies on the genome. By using the rDNA fragment as a targeting DNA on the expression vector; the objective DNA to be expressed on said vector can be integrated into the host genome, and also can exist as multicopies. This may give the gene dosage effects which will contribute to the overexpression of the objective enzymes. In the Examples of the present invention, such rDNA fragment was conveniently used for this purpose. The invention is intended to include other forms of expression vectors which serve equivalent functions and which are, or subsequently become, known.

An isolated DNA sequence encoding β-carotene ketolase may be manipulated in a variety of ways to provide for expression of the polypeptide. Manipulation of the nucleotide sequence encoding said β-carotene ketolase prior to its insertion into an expression vector may be desirable or necessary depending on the expression vector. The techniques for modifying nucleotide sequences utilizing cloning methods are well known in the art.

Recombinant DNA consisting of said gene of β-carotene ketolase which was cloned in the expression vector will be introduced into a host microorganism. Methods for the introduction of foreign DNA into fungal cells including a microorganism belonging to Phaffia are well known in the art. These include, for example, transformation by the LiCl method, protoplast fusion, electroporation, particle-gun methods which comprises bombardment by particles coated with DNAs, and other methods known in the art. In the Examples of the present invention, the particle-gun method was applied as a transformation method for P. Rhodozyma. Procedures for the particle-gun method are well known to the person killed in the art.

The recombinant organism thus obtained is capable of overexpressing the DNA sequence encoding β-carotene ketolase. Thus, the recombinant organism of the present invention is useful in the production process of xanthophyll carotenoids, especially canthaxanthin and echinenone.

A further aspect of the present invention is a biological process for producing canthaxanthin and echinenone which comprises cultivating the recombinant microorganism of Phaffia in the presence of substrate for producing carotenoids in an aqueous nutrient medium under aerobic conditions, and isolating the resulted carotenoids from the cells of said recombinant microorganism or from the cultured broth.

Carotenoids including the xanthophylls are normally produced by cultivating a strain of Phaffia in a medium which comprises suitable macro- and micronutrients for the cells, such as molasses, saccharose or glucose as a carbohydrate source for cell growth and also as a substrate for producing carotenoids, and nitrogen sources such as corn steep liquor, yeast extract, diammonium sulphate, ammonium phosphate, ammonium hydroxide or urea, phosphorus sources such as ammonium phosphate and phosphoric acid and added micronutrients or mineral salts such as magnesium sulphate, zinc sulphate and biotin or desthiobiotin.

The preferable conditions for cultivation are a pH in the range of from 4 to 8 and a temperature in the range of from 15 to 26° C. for 24 to 500 hours. More preferable conditions for cultivation are a pH in the range of from 5 to 7 and a temperature in the range of from 18 to 22° C. for 48 to 350 hours.

In the cultivation, aeration and agitation usually give favorable results for the production of carotenoids.

Once carotenoids are produced by cultivating the recombinant strain of Phaffia using the methods of the present invention, the carotenoids can be isolated either from the medium, in the case they are secreted into the medium, or from the cells of the microorganism and, if necessary, separated from other carotenoids that may be present in case one specific carotenoid is desired, by methods known in the art.

Carotenoids produced in accordance with the present invention can be used in a process for the preparation of food or feed. A man skilled in the art is familiar with such processes. Such compound food or feed can further comprise additives or components generally used for such purpose and known in the state of the art.

The following Examples further illustrate the present invention, but these are not thereby limiting the scope of the invention.

The following materials and methods were employed in the Example described below:

Strains

P. rhodozyma ATCC96594 (re-deposited under the accession No. ATCC 74438 on Apr. 8, 1998 pursuant to the Budapest Treaty)

P. rhodozyma ATCC96815 (re-deposited under the accession No. ATCC 74486 on Feb. 18, 1999 pursuant to the Budapest Treaty)

E. coli TOP10: F⁻, mcrA, delta(mrr-hsdRMS-mcrBC), phi80, delta(lacZ M15), delta(lacX74), recA1, deoR, araD139, (ara-leu)7697, galU, galK, rpsL (Str^{r}), endA1, nupG (Invitrogen Corporation, Carlsbad, USA)

Vectors

pCR2.1-TOPO (Invitrogen Corporation, Carlsbad, USA)

pGEM-T (Promega Corporation, USA)

Methods

Restriction enzymes and T4 DNA ligase were purchased from Takara Shuzo (Ohtsu, JP). Polymerase chain reaction (PCR) was performed with the thermal cycler from Perkin Elmer model 2400. Each PCR condition is described in examples. PCR primers were purchased from a commercial supplier. Fluorescent DNA primers for DNA sequencing were purchased from Pharmacia. DNA sequencing was performed with the automated fluorescent DNA sequencer (ALFred, Pharmacia).

Authentic sample for β-carotene was purchased from WAKO (Osaka, Japan). Canthaxanthin and echinenone were obtained from Roche Vitamins AG (Basle, Switzerland).

### EXAMPLE 1

### Preparation of Components of the Expression Vector

The components of the expression vector, a G418 resistant gene, promoter and terminator region of glyceraldehyde-3-phosphate dehydrogenase gene (hereinafter referred to as GAP) of P. rhodozyma, and rDNA fragment of P. rhodozyma were prepared. A G418 resistant gene cassette was prepared as follows. A Sac I-linker was ligated into the unique Hind III site of the vector, pUC-G418 (U.S. Pat. No. 6,365,386 B 1), which was harboring the G418 resistant gene cassette, and resulted vector was named as pG418Sa512. A 1.7 kb Kpn I/Sac I fragment cut out from pG418Sa512 was used as a G418 resistant gene cassette.

Each of the promoter and the terminator of GAP gene and the rDNA fragment was obtained by PCR using the gehomic DNA of P. rhodozyma ATCC 96594 as template. To obtain the genomic DNA, a QIAGEN Blood & Cell Culture DNA Midi Kit (QIAGEN, Germany) was used with the cells of P. rhodozyma ATCC 96594 obtained by overnight culture in YPD (Difco Laboratories) medium.

Using the prepared genomic DNA as a template, PCR was performed using an Advantage-HF PCR Kit (CLONTECH Laboratories, Inc., USA) and a thermal cycler (Perkin Elmer 2400, USA).

The synthetic primers used to amplify the promoter sequence of GAP were: GAP#1 (SEQ ID NO: 1)(having a Not I site GCGGCCGC) and GAP#5 (SEQ ID NO:2) (having a Sma I site CCCGGGG).

The initial template denaturation step consisted of 5 min at 94° C. An amplification cycle of 30 seconds at 94° C., 30 seconds at 55° C., and 1 min at 72° C. was repeated for 25 times. After additional 10 min reaction at 72° C., the reaction mixture was kept at 4° C. By this reaction, DNA fragment containing the GAP promoter (398 bp) was amplified. This amplified GAP promoter was ligated with a pCR2.1-TOPO vector and introduced into E.coli TOP 10 cells by using a TOPO TA Cloning kit (Invitrogen Corporation, USA). Several clones were selected for sequence analysis. The sequence of the cloned GAP promoter of each candidate clone was examined. One of the DNA clones that showed completely the same sequence as the GAP promoter of P. rhodozyma (GenBank accession No. Y08366) was named as pTOPO-pGAP2#1. A 398 bp Not I/Sma I fragment cut out from pTOPO-pGAP2#1 was used as the GAP promoter cassette.

The two synthetic primers used to amplify the terminator sequence of GAP were: GAP#33 (SEQ ID NO:3)(having a BamH I-Sal I site GGATCCGTCGAC) and GAP#4 (SEQ ID NO:4) (having a Kpn I site GGTACC).

The PCR conditions were the same as those for the GAP promoter described above. By this reaction, DNA fragment containing the GAP terminator (302 bp) was amplified. This amplified GAP terminator was ligated with a pCR2.1-TOPO vector and introduced into E.coli TOP 10 cells by using the TOPO TA Cloning kit. Several clones were selected for sequence analysis. The sequence of the cloned GAP terminator of each candidate clone was examined. One of the DNA clones that showed completely the same sequence as the GAP terminator of P. rhodozyma (GenBank accession No. Y08366) was named as pTOPO-tGAP#1. A 302 bp BamH I/Kpn I fragment which was cut out from pTOPO-tGAP#1 was used as the GAP terminator cassette.

The two synthetic primers used to amplify the rDNA fragment were: R#1 (SEQ ID NO:5) (having a Sac I site GAGCTC) and R#2 (SEQ ID NO:6)(having a Not I-Sac I site GCGGCCGCGAGCTC).

The PCR conditions were the same as those for the GAP promoter described above. By this reaction, DNA fragment containing the rDNA (3126 bp) was amplified. This amplified rDNA was ligated with a pCR2.1-TOPO vector and introduced into E.coli TOP 10 cells by using the TOPO TA Cloning kit. Several clones were selected for sequence analysis. The sequence of the cloned the rDNA of each candidate clone was examined. One of the DNA clones that showed completely the same sequence as the rDNA of P. rhodozyma (GenBank accession No. D31656, AF139632) was named as pTOPO-rDNA#1. A 1960 bp Sac II/Not I fragment cut out from pTOPO-rDNA#1 was used as the rDNA cassette.

### EXAMPLE 2

Construction of the expression vector carrying the β-carotene ketolase gene (crtW) and use for the production of canthaxanthin and echinenone

PCR based gene synthesis was applied to obtain the nucleotide sequence of the artificial crtW gene encoding the β-carotene ketolase of Alcaligenes strain PC-1 by back translating the amino acid sequence (GenBank accession No. D58422). The detailed methods are described in the Example in U.S. Pat. No. 6,124,113. In this case, each of two terminal primers are designed to introduce the restriction site of Sma I and BamH I to the 5′-end and 3′-end of the crtW gene, respectively.

The expression vector carrying the crtW gene was constructed by ligating the rDNA cassette and the GAP promoter cassette (obtained in Example 1), the crtW gene constructed as described above, and the GAP terminator cassette and the G418 resistant gene cassette (obtained in Example 1) to be aligned in this order using a pGEM-T as a backbone.

The resulting expression vector was introduced into P. rhodozyma ATCC 96815 by using the particle-gun methods as described in EP 1,158 051.

The crtW-recombinant strains of P. rhodozyma ATCC 96815 thus obtained are cultivated in 50 ml production medium in 500 ml Erlenmeyer flask with shaking at 20° C. for 7 days after inoculation of seed culture prepared in 7 ml seed medium in test tube (21 mm in diameter) with shaking at 20° C. for 3 days. Appropriate volume of the culture broth was withdrawn and used for analysis of the cell growth and the productivity of carotenoids.

Medium composition was as follows:

Seed medium: Glucose 30.0 g/l, NH₄Cl 4.83 g/l, KH₂PO₄ 1.0 g/l, MgSO₄-7H₂O 0.88 g/l, NaCl 0.06 g/l, CaCl₂-2H₂O 0.2 g/l, KH phtalate 20.0 g/l, FeSO₄-7H₂O 28 mg/l, Trace element solution 0.3 ml, Vitamin stock solution 1.5 ml, (pH was adjusted at 5.4-5.6)

Trace element solution: 4N H₂SO₄ 100 ml/l, citric acid-H₂O 50.0 g/l, ZnSO₄-7H₂O 16.7 g/l, CuSO₄-5H₂O 2.5 g/l, MnSO₄-4,5H₂O 2.0 g/l, H₃BO₃ 2.0 g/l, Na₂MoO₄ 2.0 g/l, KI 0.5 g/l

Vitamin stock solution for seed medium: 4N-H₂SO₄ 17.5 ml/l, myo-Inositol 40.0 g/l, Nicotinic acid 2.0 g/l, Ca-D-pantothenate 2.0 g/l, Vitamin B₁ (thiamin HCl) 2.0 g/l, p-Aminobenzoic acid 1.2 g/l, Vitamin B₆ (pyridoxine HCl) 0.2 g/l, Biotin stock solution 8.0 ml

Biotin stock solution was prepared by the addition of 4N-H₂SO₄ to 50 ml of ethanol to a total of 100 ml. Then, 400 mg of D-biotin was added.

Production medium: Glucose 22.0 g/l, KH₂PO₄ 14.25 g/l, MgSO₄-7H₂O 2.1 g/l, CaCl₂-2H₂O 0.865 g/l, (NH₄)₂SO₄ 3.7 g/l, FeSO₄-7H₂O 0.28 g/l, Trace element solution 4.2 ml, Vitamin stock solution 9.35 ml, (pH was adjusted at 5.5)

Vitamin stock solution for production medium: 4N H₂SO₄ 17.5 ml/l, Nicotinic acid 2.0 g/l, Ca-D-pantothenate 3.0 g/l, Vitamin B₁ (thiamin HCl) 2.0 g/l, p-Aminobenzoic acid 1.2 g/l, Vitamin B₆ (pyridoxine HCl) 0.2 g/l, Biotin stock solution 30.0 ml

A portion of the seed culture broth (2.5 ml) was transferred to 47.5 ml of the production medium in 500 ml Erlenmeyer flask. Then the cultivation was started at 20° C. with rotary shaking at 200 rpm. At the second day of the fermentation, 5 ml of 50% glucose solution was added to the medium and the fermentation continued. At the fourth day of the fermentation, 2 ml of cultured broth were withdrawn and 5 ml of 50% glucose solution added again to the medium, and cultivation was continued for additional 3 days. At the seventh day, an aliquot of the culture was withdrawn and used for analysis of the carotenoids production and the cell growth.

For analysis of the cell growth, optical density at 660 nm was measured by using UV-1200 photometer (Shimadzu Corp., Kyoto, Japan).

For analysis of the content of β-carotene, canthaxanthin and echinenone, the withdrawn broth was mixed with a solvent mixture (ethyl alcohol, hexane and ethyl acetate) and carotenoids were extracted from the broth and the cells of P. rhodozyma by vigorous shaking with glass beads. After extraction, disrupted cells and glass beads were removed by centrifugation and the resultant supernatant was analyzed by HPLC for the carotenoids content. The HPLC conditions used were as follows: HPLC column: Chrompack Lichrosorb si-60 (4.6 mm, 250 mm); Temperature: room temperature; Eluent: acetone/hexane (18/82) add 1 ml/l of water to eluent; Injection volume: 10 μl; Flow rate: 2.0 ml/min; Detection: UV at 450 nm

1. A process for producing canthaxanthin and echinenone which comprises cultivating a recombinant microorganism which is expressing a β-carotene ketolase gene and belonging to the genus Xanthophyllomyces (Phaffia) and which accumulates β-carotene in an aqueous nutrient medium under aerobic conditions, and isolating the resulted carotenoids from the cells of said recombinant microorganism or from the cultured broth, wherein the β-carotene ketolase gene is originated from a microorganism which is selected from the group consisting of microorganisms of the genera Agrobacterium, Alcaligenes, Paracoccus and Haematococcus having the β-carotene ketolase gene.

2. The process according to claim 1, wherein the recombinant microorganism is derived from Xanthophyllomyces dendrorhous (Phaffia rhodozyma) ATCC96815, or a mutant thereof.

3. The process according to claim 1, wherein the β-carotene ketolase gene is originated from a microorganism which is selected from the group consisting of Agrobacterium aurantiacum, Alcaligenes PC-1, Paracoccus marcusii MH1, a gram-negative bacteria E-396 (FERM BP-4283), and Haematococcus pluvialis, having the , β-carotene ketolase gene.

4. The process according to claim 1, wherein the β-carotene ketolase gene is originated from Alcaligenes PC-1 or the DNA sequence of the β-carotene ketolase gene is substantially homologous thereto.

5. The process according to claim 1, wherein the β-carotene ketolase gene is expressed in the recombinant microorganism using the control sequences.

6. The process according to claim 1, wherein the cultivation is carried out at a pH in the range of from 3 to 7 and at a temperature in the range of from 15 to 26° C. for 24 to 500 hours.

7. The process according to claim 7, wherein the cultivation is carried out at a pH in the range of from 5 to 7 and at a temperature in the range of from 18 to 22° C. for 48 to 350 hours.
